(19) United States
(12) Patent Application Publication
(10) Pub. No.: US 2015/0083124 A1
(43) Pub. Date: Mar. 26, 2015

(54) RESPIRATORY PATIENT INTERFACE DEVICE AND HEADGEAR CONNECTION APPARATUS

(71) Applicant: KONINKLIJKE PHILIPS N.V., EINDHOVEN (NL)

(72) Inventors: Lauren Patricia Chodkowski, Pittsburgh, PA (US); Robert William Baiko, Pittsburgh, PA (US); Peter Chi Fai Ho, Pittsburgh, PA (US)

(21) Appl. No.: 14/397,760

(22) PCT Filed: Apr. 23, 2013
(86) PCT No.: PCT/IB2013/053197

§ 371 Date: Oct. 29, 2014

Related U.S. Application Data
(60) Provisional application No. 61/642,152, filed on May 3, 2012.

(51) Int. Cl. A61M 16/08; A61M 16/06

(52) U.S. Cl. CPC A61M 16/0816; A61M 16/0683
USPC 128/202.27

(57) ABSTRACT

An improved connection apparatus is structured to extend between a mask (8) and a headgear (12). The headgear has a first strap (16) and a second strap (20) structured to overlie the patient's head. The connection apparatus can be generally stated as including a clip apparatus (24) and a face plate. The clip apparatus can be generally stated as including a clip (32A,B) having a first mount (44) that is structured to be connected with the first strap and a second mount (48) that is structured to be connected with the second strap. The face plate is structured to overlie at least a portion of the mask. The face plate (28) can be generally stated as including an attachment apparatus having a connection upon which the clip is structured to be disposed. At least a portion of the clip is removable from at least a portion of the connection.

### CROSS-REFERENCE TO RELATED APPLICATIONS

This patent application claims the priority benefit under 35 U.S.C. §119(e) of U.S. Provisional Application No. 61/642,152 filed on May 3, 2012, the contents of which are herein incorporated by reference.

### BACKGROUND OF THE INVENTION

1. Field of the Invention

The present invention relates generally to a respiratory patient interface device structured to provide a flow of breathing gas to a patient, and, more particularly, to a connection apparatus that enables a pair of straps of a headgear to be mounted to a clip which is, in turn, mounted on a mask of the respiratory patient interface device.

2. Description of the Related Art

There are numerous situations where it is necessary or desirable to deliver a flow of breathable gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder such as sleep apnea syndrome, in particular, obstructive sleep apnea (OSA), or congestive heart failure.

Non-invasive ventilation and pressure support therapies involve the placement of a respiratory patient interface device including a patient interface that is typically secured on the face of a patient by a headgear assembly. The patient interface may be, without limitation, a nasal mask that covers the patient's nose, a nasal cushion having nasal prongs that are received within the patient's nares, a nasal/oral mask that covers the nose and mouth, or full face mask that covers the patient's face. It is known to maintain such devices on the face of a wearer by a headgear having one or more straps adapted to fit over/around the patient's head. Because such respiratory patient interface devices are typically worn for an extended period of time, it is important for the headgear to maintain the patient interface in a desired position while doing so in a manner that is comfortable to the patient.

Such respiratory patient interface devices typically must also form a reliable and generally fluid-tight seal with the face of the patient in the vicinity of the airways in order to ensure that the flow of air is delivered to the airways and does not leak from around the patient interface. Due to the great variability of the facial features of the various patients who require such therapy, reliable seals have sometimes been difficult to provide and/or maintain.

Previously known devices have sometimes been difficult to use on an ongoing basis due to the difficulty inherent in adjusting multiple straps of a headgear with respect to a mask. For example, and as is indicated generally in FIG. 1, a previously known respiratory patient interface device A is depicted as being situated on a patient C and as including a mask E that is connected with a headgear G. The headgear G includes a first strap I that extends over the temporal region of the head of the patient C and a second strap J that extends over the occipital region of the head of patient C. An elastic tie element M extends between the first and second straps I and J and resists movement of the first strap I in a direction generally toward the eye of patient C. However, the first and second straps I and J each have an adjustable connection (such as through the use of hook and loop fasteners) with the left side of mask E, and headgear G further includes another set of first and second straps (not expressly depicted herein) that likewise connect with the opposite side (i.e., the right side) of mask E for a total of four adjustable connections between headgear G and mask E.

While all four of the straps of headgear G need not necessarily be detached from mask E in order to remove respiratory patient interface device A from patient C, the interplay of four adjustable straps that connect with mask E can make the proper adjustment of respiratory patient interface device A for comfort and fit tedious and sometimes difficult.

### SUMMARY OF THE INVENTION

In certain embodiments, the general nature of the invention can be stated as including an improved connection apparatus that is structured to extend between a mask and a headgear. The mask is structured to provide a flow of breathing gas to a patient. The headgear has a first strap and a second strap, at least a portion of the first strap being structured to overlie at least one region of the patient's head, and at least a portion of the second strap being structured to overlie at least one other region of the patient's head. The connection apparatus can be generally stated as including a clip apparatus and a face plate. The clip apparatus can be generally stated as including a clip having a first mount that is structured to be connected with the first strap and a second mount that is structured to be connected with the second strap. The face plate is structured to overlie at least a portion of the mask. The face plate can be generally stated as including an attachment apparatus having a connection upon which the clip is structured to be disposed. At least a portion of the clip is removable from at least a portion of the connection.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is, as mentioned above, a depiction of a prior art respiratory patient interface device installed on a patient;

FIG. 2 is a side elevational view of an improved respiratory patient interface device that comprises an improved connection apparatus in accordance with a first embodiment of the disclosed and claimed concept;

FIG. 3 is a side view of a portion of the connection apparatus of FIG. 2;

FIG. 4 is a top plan view of the connection apparatus of FIG. 2;

FIG. 5 is a side elevational view of an improved respiratory patient interface device that includes an improved connection apparatus in accordance with a second embodiment of the disclosed and claimed concept;

FIG. 6 is a side elevational view of an improved respiratory patient interface device that includes an improved connection apparatus in accordance with a third embodiment of the disclosed and claimed concept;

FIG. 7 is a sectional view as taken along line 7-7 of FIG. 6;

FIG. 8 is an exploded side elevational view of an improved respiratory patient interface device that includes an improved connection apparatus in accordance with a fourth embodiment of the disclosed and claimed concept;

FIG. 9 is a top plan view of a portion of the connection apparatus of FIG. 8;

FIG. 10 is a front elevational view of a clip the improved connection apparatus of FIG. 8;

FIG. 11 is a side elevational view of an improved respiratory patient interface device having an improved connection apparatus in accordance with a fifth embodiment of the disclosed and claimed concept;

FIG. 12 is a side elevational view of an improved respiratory patient interface device having an improved connection apparatus in accordance with a sixth embodiment of the disclosed and claimed concept;

FIG. 13 is a side elevational view of an improved respiratory patient interface device having an improved connection apparatus in accordance with a seventh embodiment of the disclosed and claimed concept; and

FIG. 14 is a side elevational view of an improved respiratory patient interface device having an improved connection apparatus in accordance with an eighth embodiment of the disclosed and claimed concept.

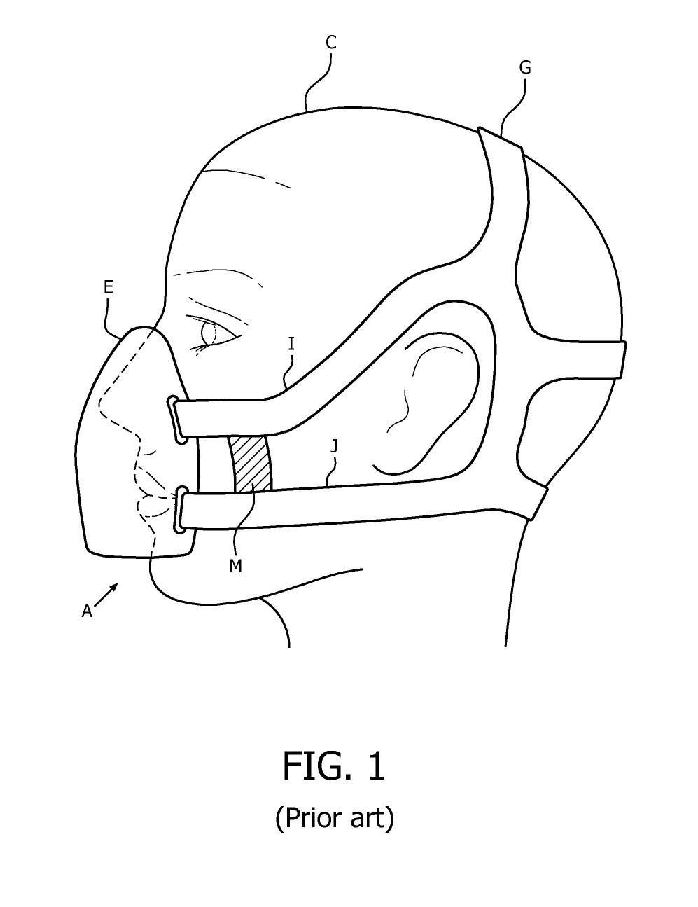

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

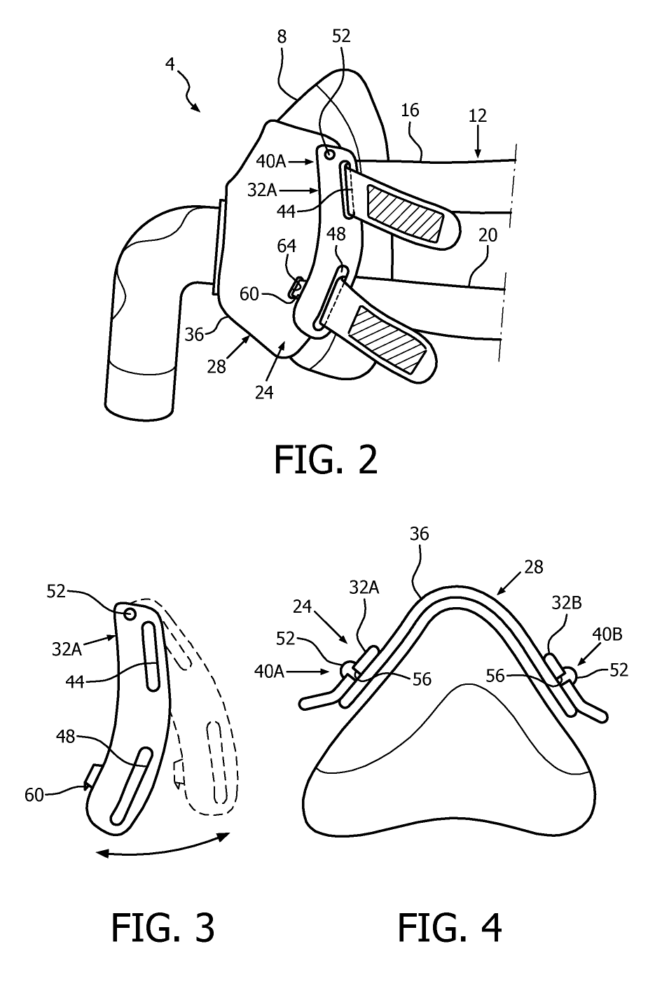

As can be seen in FIG. 2, an improved respiratory patient interface device in accordance with the disclosed and claimed concept includes an improved connection apparatus 4 that extends between a mask 8 and a headgear 12. As is generally understood in the relevant art, mask 8 is configured to be engaged with the face of a patient and to provide a flow of breathing gas to the airways of the patient. Headgear 12 includes a first strap 16 and a second strap 20 that are adjustably connected with connection apparatus 4. In the depicted exemplary embodiment, first and second straps 16 and 20 are adjusted through the use of hook and loop fasteners, although other adjustment structures can be employed with departing from the present concept. As will be set forth in greater detail below, connection apparatus 4 advantageously enables mask 8 and/or headgear 12 to be removed from the patient without the need to individually remove or readjust either of first and second straps 16 and 20.

More particularly, connection apparatus 4 can be said to include a clip apparatus 24 and a face plate apparatus 28. As can be understood from FIGS. 2-4, face plate apparatus 28 is disposed on and at least partially overlies mask 8 whereas headgear 12 is connected with clip apparatus 24. As will be set forth in greater detail below, the advantageous interplay between clip apparatus 24 and face plate apparatus 28 advantageously enables headgear 12 to be moved with respect to mask 8 without the need to individually remove either of first and second straps 16 and 20 from connection apparatus 4, which facilitates such removal and also facilitates the reinstallation of the respiratory patient interface device on the patient.

Clip apparatus 24 can be said to include a pair of clips 32A and 32B, it being noted that only clip 32A is expressly depicted in FIG. 2. Face plate apparatus 28 can be said to include a face plate 36 and a pair of attachment apparatuses 40A and 40B. Attachment apparatus 40A is cooperable with clip 32A, and attachment apparatus 40B is cooperable with clip 32B. Clips 32A and 32B each have a first mount 44 and a second mount 48 that are cooperable with first and second straps 16 and 20, respectively. In the depicted exemplary embodiment, first and second mounts 44 and 48 are each in the form of an elongated receptacle that are structured to receive one of first and second straps 16 and 20 therein. While the interaction between first and second straps 16 and 20 is depicted in FIG. 2 only with respect to clip 32A, it is understood that headgear 12 has a pair of straps that are likewise each adjustably connected with first and second mounts 44 and 48 of clip 32B.

Attachment apparatuses 40A and 40B can be said to each include a pivotable connection element in the exemplary form of a pivot pin 52 that is situated on face plate 36 and that is pivotably receivable in a hole 56 (FIG. 4) formed in each of clips 32A and 32B. Clips 32A and 32B are thus pivotable with respect to face plate 36 about pivot pins 52.

Attachment apparatuses 40A and 40B further each include a fixed connection element in the exemplary form of a hook-shaped locking tab 60 situated on each of clips 32A and 32B and which are each receivable in one a pair of apertures 64 formed in face plate 36 on opposite sides thereof (it being noted that only the aperture 64 that is situated on the left side of face plate 38 is expressly depicted herein, it being further noted that the aperture 64 situated on the right side of face plate 38 is in a mirror image location as that depicted in FIG. 2).

While attachment apparatuses 40A and 40B are discussed in the hierarchy herein as being a part of face plate apparatus 28, it is noted attachment apparatuses 40A and 40B are intended to enable cooperation between face plate 36 and clips 32A and 32B. As such, certain of the various components of attachment apparatuses 40A and 40B may be situated on face plate 36, and certain of the various components of attachment apparatuses 40A and 40B may be situated on clips 32A and 32B, and such relationship may be changed from what is expressly depicted herein without departing from the scope of the present concept. It thus is expressly noted that in other embodiments than those expressly depicted herein the various arrangements of elements can be varied without departing from the scope of the disclosed and claimed concepts.

As can be understood from FIG. 2, when locking tab 60 is received in aperture 64, clip 32A is retained in a generally fixed relation with respect to face plate 36, and thus first and second straps 16 and 20 are retained in a fixed relationship with respect to mask 8. Such a condition can be considered to be a locked condition of attachment apparatus 40A. However, and as can be understood from FIG. 3, when locking tab 60 is released from aperture 64, clip 32A is pivotable about pivot pin 52, which can be considered to be an unlocked condition of attachment apparatus 40A. The same can be said for clip 32B.

In the unlocked condition of attachment apparatus 40A, and the corresponding (but not expressly depicted herein) unlocked condition of attachment apparatus 40B, first and second straps 16 and 20 and thus headgear 12 are permitted to move with respect to mask 8, which permits mask 8 and/or headgear 12 to be removed from the patient. Advantageously, however, such removal of mask 8 and/or headgear 12 occurs without removing or adjusting either of first and second straps 16 and 20. Rather, first and second straps 16 and 20 remain connected with clip 32A, and clip 32A is released from its fixed relationship with face plate 36 and is pivotable with respect to face plate 36. As such, mask 8 and/or headgear 12 can be removed from the patient without disturbing the adjustment (of first and second straps 16 and 20) for fit and comfort that had already been achieved. Similarly, mask 8 and headgear 12 can be reinstalled on the patient and the locking tabs 60 returned to apertures 64 in order to reinstall the respiratory patient interface device on the patient. Such reinstallation of mask 8 and headgear 12 on the patient without the need to adjust either of first and second straps 16 and 20 pursuant to such removal and/or replacement results in the significant time savings to the patient, which is highly desirable. Moreover, since the fit of mask and headgear 8 and 12 on the patient can be repeatedly replicated without the need for readjustment, comfort and fit can generally be said to be improved as a result.

Much of the foregoing discussion has been in terms of clip 32A, and it is understood that in the depicted exemplary embodiment clips 32A and 32B are mirror images of one another, as are attachment apparatuses 40A and 40B. In removing mask 8 and/or headgear 12 from the patient, the patient can move either or both of clips 32A and 32B from the locked condition to the unlocked condition and pivot either or both of them with respect to face plate 36, after which mask 8 and/or headgear 12 can be removed from the patient.

It is understood, however, that such removal potentially can be performed by moving only a single one of clips 32A and 32B to the unlocked condition and pivoting it with respect to face plate 36. In this regard, therefore, it is understood that alternative embodiments of connection apparatus 4 potentially may be configured to have only a single clip, such as clip 32A, with first and second straps 16 and 20 of headgear 12 mounted thereto. Such a clip may be situated at either the left side or the right side of mask 8, and the straps at the side of headgear 12 opposite the clip may connect directly with mask 8 and/or face plate 36. As such, it is expressly noted that the pair of clips 32A and 32B are provided herein in an exemplary fashion and for purposes of convenience to the user, and it is thus also understood that the advantageous results provided herein can be achieved through the potential use of only a single one of clips 32A and 32B.

Moreover, further embodiments are set forth below and are described generally in terms only of the left side of a respiratory patient interface device. For each of the additional embodiments set forth below, it is understood that such embodiments typically will have similar structures at the right side of the respiratory patient interface device, but that such structures are not expressly described herein for purposes of simplicity of disclosure. It is likewise understood, however, that the additional embodiments below can potentially be configured to include such structures at only one side of such respiratory patient interface device and can still provide the advantageous benefits described herein.

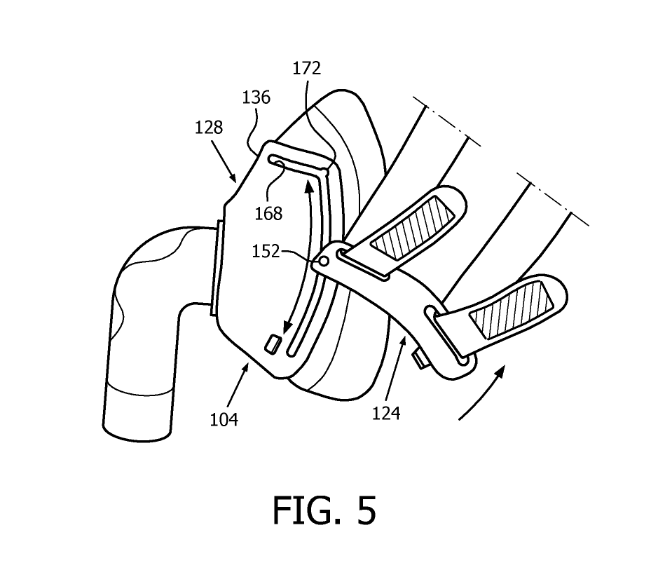

FIG. 5 depicts another improved respiratory patient interface device that includes an improved connection apparatus 104 in accordance with a second embodiment of the present invention. Connection apparatus 104 is similar to connection apparatus 4 in that it includes a clip apparatus 124 and a face plate apparatus 128 that are cooperable and that are movable between locked and unlocked conditions. However, face plate apparatus 128 includes a face plate 136 that further has an elongated slot 168 formed therein within which pivot pin 152 is slidably situated. That is, clip apparatus 124 is pivotable about pivot pin 152, and pivot pin 152 is additionally slidable within slot 168. Slot 168 provides for improved range of movement of a headgear with respect to a mask in the unlocked condition, which is advantageous.

Moreover, slot 168 has a seat 172 formed therein that is structured to receive pivot pin 152 when clip apparatus 124 is in its locked condition. That is, while connection apparatus 104 is depicted herein only in its unlocked condition, it can be understood that in moving connection apparatus 104 to its locked condition, pivot pin 152 is received against seat 172, and the locking tab of clip apparatus 124 is received in the aperture formed in face plate 136. In such a condition, both pivoting and sliding movement of pivot pin 152 with respect to face plate 136 is resisted.

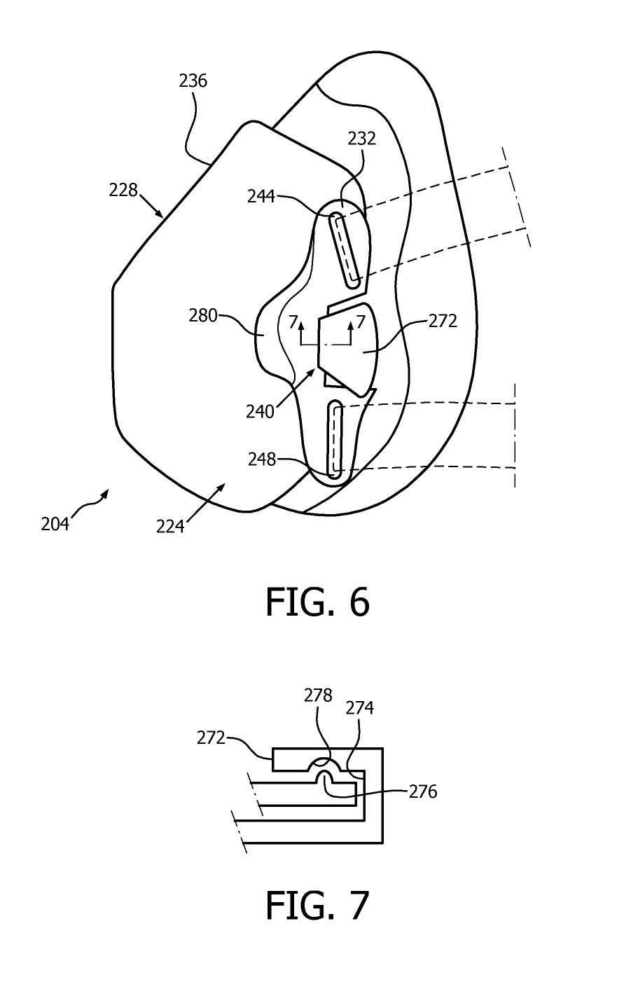

An improved respiratory patient interface device having an improved connection apparatus 204 in accordance with a third embodiment of the present invention is depicted generally in FIGS. 6 and 7 in conjunction with another mask and headgear. Connection apparatus 204 includes a clip apparatus 224 having a clip 236, and further includes a face plate apparatus 248 having a face plate 236. Face plate apparatus 228 further includes an attachment apparatus 240. While attachment apparatus 240 is similar to attachment apparatus 40A, some differences exist in that attachment apparatus 240 is configured as a seat 272 having a receptacle 274 that is structured to lockably and releasably receive clip 232 therein. Specifically, and as can be seen from FIG. 7, clip 232 and receptacle 274 have cooperative structures that enable such locking and removability and which, in the depicted exemplary embodiment, include a protrusion 276 situated on clip 232 and a depression 278 formed in seat 272. As can be understood from FIG. 7, protrusion 276 is received in depression 278 to lock clip 232 with respect to seat 272. However, clip 232 is also provided with a finger grip 280 which permits the user to grasp clip 232 and pull it from receptacle 274 by overcoming the frictional forces between protrusion 276 and depression 278 and to detach clip 232 as a whole from seat 272. Such removal enables the mask and/or the headgear to be removed from the patient. Clip 232 can be returned to seat 272 in a reverse fashion.

As can be understood from FIG. 6, the two straps of the headgear are received in first and second mounts 244 and 248, respectively, which are situated at alternate sides of seat 272 when clip 232 is received in receptacle 274 of seat 272. Moreover, it can be understood that finger grip 280 is generally in an anterior position with respect to seat 272 and first and second mounts 244 and 248 when the respiratory patient interface device is mounted on a patient.

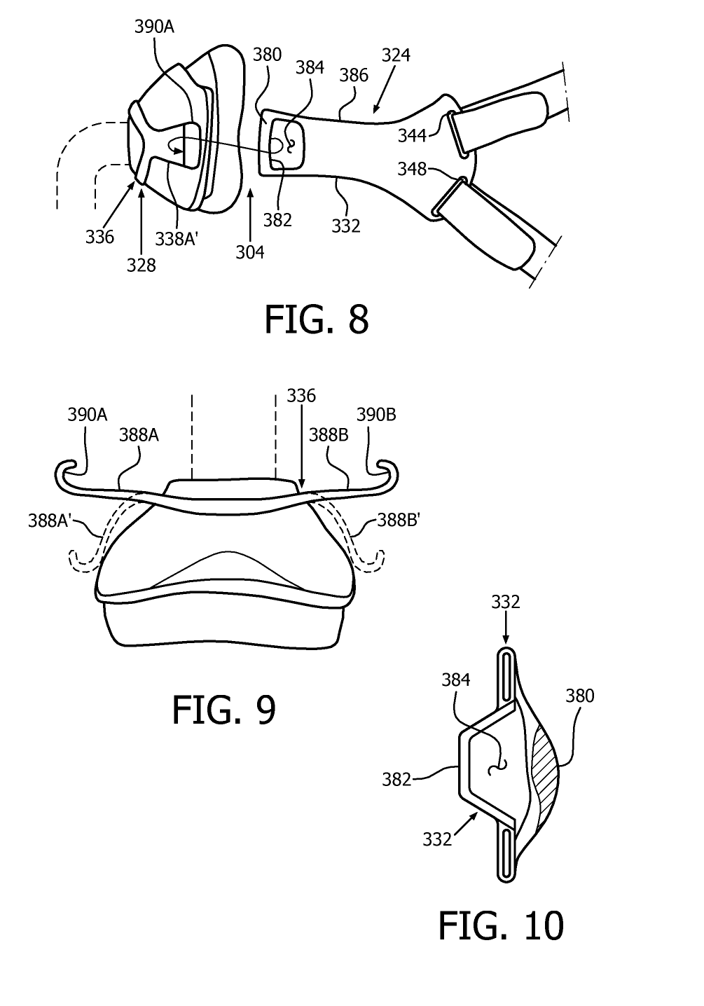

As can be understood from FIGS. 8-10, an improved respiratory patient interface device having an improved connection apparatus 304 in accordance with a fourth embodiment of the present invention is structured to extend between a mask and a headgear and to be a part of an improved respiratory patient interface device for use on a patient. Connection apparatus 304 can be said to include a clip apparatus 324 and a face plate apparatus 328 that are cooperable with one another. Clip apparatus 324 includes a clip 332, and face plate apparatus 328 includes a face plate 336. Clip 332 can be said to include a base 382 adjacent an opening 384 and to further include an elongated lug 386 having a first mount 344 and a second mount 348 formed in lug 386 at a location distal to base 382. It is understood that the exemplary embodiment of connection apparatus 304 actually includes a pair of clips 332, although only a single instance of clip 332 is depicted herein for purposes of simplicity.

As can be understood from FIG. 9, face plate 336 includes a pair of legs 388A and 388B that each have a seat 390A and 390B, respectively, formed therein at the free end thereof. It can be understood from FIG. 8 that base 382 of clip 332 is lockably and removably received in seat 390A, and should be clear to one of ordinary skill in the relevant art the other instance of clip 332 that is not expressly depicted herein is likewise lockably and removably receivable in seat 390B.

As can further be understood from FIG. 9, legs 388A and 388B are movable between a free state (which exists when clips 332 are removed from seats 390A and 390B) and a deflected state (as is indicated in dashed lines at the numerals 388A′ and 388B′, although clips 332 are not expressly depicted in FIG. 9 for purposes of simplicity of disclosure). Seats 390A and 390B in their free state are situated in a direction generally anterior to the position of such seats 390A and 390B in the deflected position of legs 388A and 388B (depicted in dashed lines at the numerals 388A′ and 388B′ in FIG. 9). In this regard, it is noted that FIG. 8 depicts the deflected state of leg 388A′, albeit with clip 332 removed from seat 390A for purposes of illustration.

When base 382 is received in seat 390A, the two are lockably connected together. Such connection can potentially be made pivotable if base 382 is of a circular profile in cross-section, although such a feature is merely optional.

Moreover, clip 332 can be removed from seat 390A by the user pulling finger grip 380 to release base 382 from seat 390A. Upon the release of clip 332 from seat 390A, leg 388A returns to its free state as is indicated generally in FIG. 9, which is in a direction anterior to the position of leg 388A when in the deflected position and situated on a patient. In this regard, therefore, it can be understood that legs 388A and 388B in their free state are relatively more visible to the patient than in their deflected position by virtue of their being protruded in the free state in a generally anterior direction with respect to the patient. Such positioning of legs 388A and 388B facilitates the reattachment of clips 332 thereto at the appropriate time.

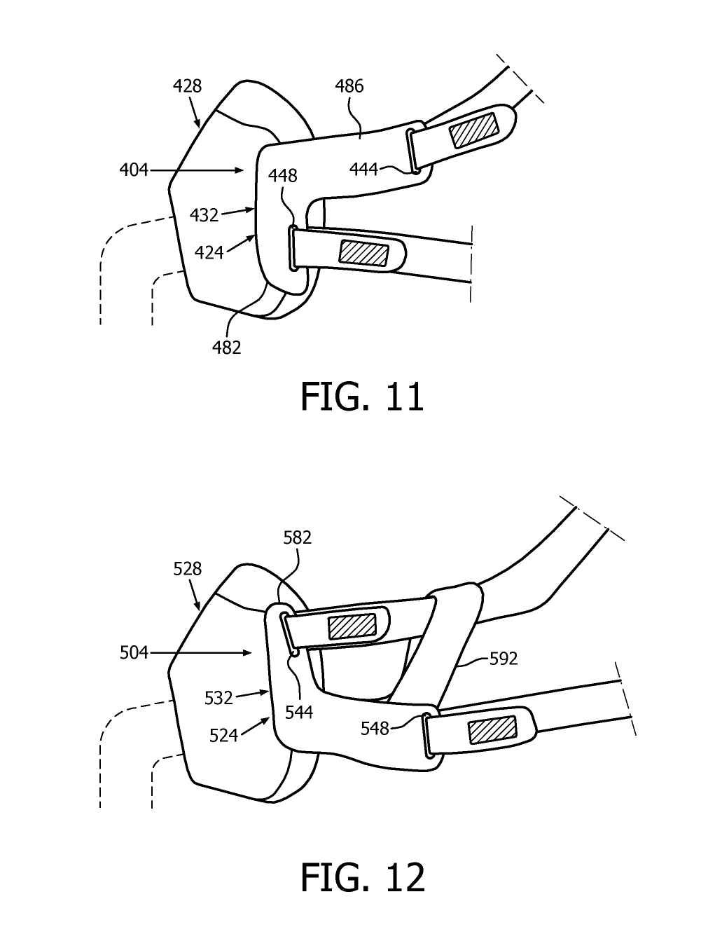

An improved respiratory patient interface device having an improved connection apparatus 404 in accordance with a fifth embodiment of the disclosed and claimed concept is depicted generally in FIG. 11. Connection apparatus 404 is an alternative connection apparatus that includes certain features that can be incorporated into an improved respiratory patient interface device in accordance with most any of the foregoing embodiments. Connection apparatus 404 includes a clip apparatus 424 and a face plate apparatus 428, with clip apparatus 424 having a clip 432 that includes a base 482 and a lug 386. The lug 386 extends in a generally posterior direction with respect to base 482 when the mask and headgear are situated on a patient. In the depicted exemplary embodiment, first mount 444 is situated on lug 486, and a second mount 444 is situated on base 482. In the depicted exemplary embodiment of connection apparatus 404, lug 486 is connected with the one of the straps of the headgear that is situated superior to the other of the straps of the headgear when the respiratory patient interface device with connection apparatus 404 is mounted on the patient. First mount 444 is situated in a generally posterior direction compared with second mount 448 when the respiratory patient interface device is situated on a patient, and lug 486 thus resists the headgear strap that is connected therewith from moving in a direction toward the eye of the patient, which is desirable.

Another embodiment of the connection apparatus in accordance with a sixth embodiment of the disclosed and claimed concept is depicted generally in FIG. 12 at the numeral 504. Connection apparatus 504 is an alternative connection apparatus that includes certain features that can be incorporated into an improved respiratory patient interface device in accordance with most any of the foregoing embodiments. Connection apparatus 504 includes a clip apparatus 524 and a face plate apparatus 528, with clip apparatus 524 including a clip 532 having a base 582 and a lug 586. Connection apparatus 504 is similar to connection apparatus 404 in these respects. However, on connection apparatus 504, lug 586 is connected with the one of the straps of the headgear that is situated inferior to the other straps of the headgear. That is, a first mount 544 is situated on base 582, and a second mount 548 is formed on lug 586. Moreover, clip 532 further includes a brace 592 situated at a position distal on lug 586 that slidably receives in a pocket formed thereon the superior strap that is received in first mount 544. The connection of posterior-extending lug 586 and the connection of brace 592 with the superior strap resist movement of the superior strap in a direction generally toward the eye of the patient, which is desirable.

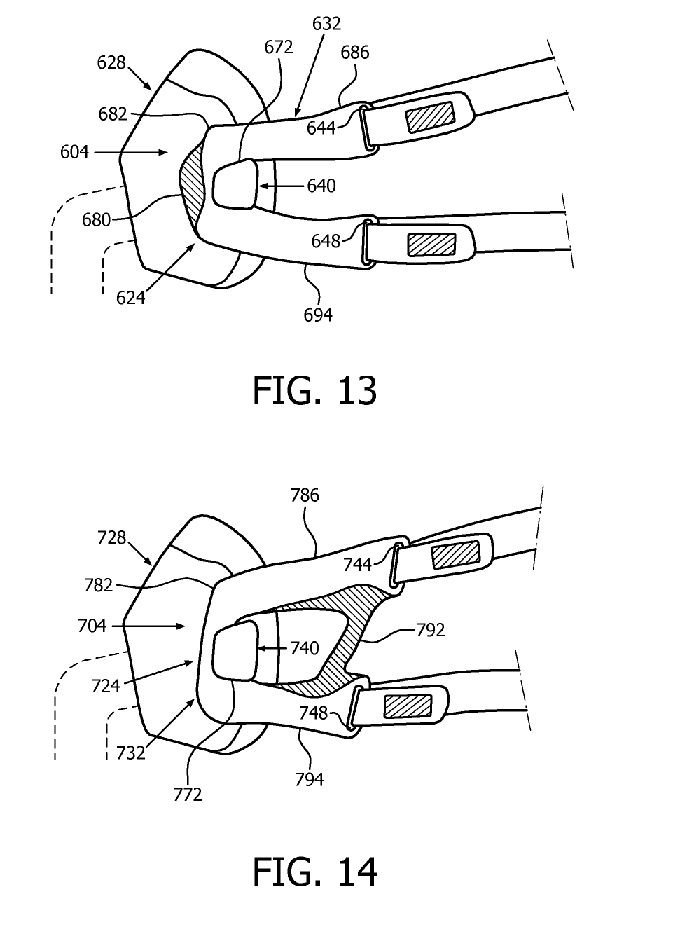

FIG. 13 depicts at the numeral 604 another connection apparatus in accordance with a seventh embodiment of the disclosed and claimed concept. Connection apparatus 604 is an alternative connection apparatus that includes certain features that can be incorporated into an improved respiratory patient interface device in accordance with most any of the foregoing embodiments. Connection apparatus 604 includes a clip apparatus 624 and a face plate apparatus 628 that are cooperable with one another, with face plate apparatus 628 including an attachment apparatus 640 that comprises a seat 672. Clip apparatus 624 comprises a clip 632 having a base 682 that is receivable on seat 672. Clip 632 further includes a first lug 686 that extends in a posterior direction from base 682 and which has a first mount 644 formed at a distal end thereof. Moreover, clip 632 includes a second lug 694 that likewise extends in a generally posterior direction from base 682 and which has a second mount 648 formed at a distal end thereon. (The posterior direction is mentioned with respect to the connection apparatus 604 being mounted on a patient.) Base 682 is lockably receivable on seat 672 and is removable therefrom by the patient with the use of a finger grip 680. Lockable retention of clip 632 on seat 672 retains first and second lugs 686 and 694 in a generally fixed position with respect to the mask and thus resists movement of both the superior and the inferior straps of the headgear in a direction generally toward the eye of the patient, which is desirable.

A further connection apparatus in accordance with an eighth embodiment of the disclosed and claimed concept is depicted generally in FIG. 14 at the numeral 704. Connection apparatus 704 is an alternative connection apparatus that includes certain features that can be incorporated into an improved respiratory patient interface device in accordance with most any of the foregoing embodiments. Connection apparatus 704 is similar to connection apparatus 604 in that connection apparatus 704 includes a clip apparatus 724 and a face plate apparatus 728, with the face plate apparatus 728 including an attachment apparatus 740 having a seat 772. Likewise, clip apparatus 724 includes a clip 732 having a base 782 from which extend (in a generally posterior direction with respect to a patient) a first lug 786 on which a first mount 744 is formed and a second lug 794 on which a second mount 748 is formed. Further advantageously, a brace 792 resiliently extends between first and second lugs 786 and 794. First and second lugs 786 and 794 thus can be made of a relatively flexible material such as fabric or silicone rubber material since the tension among the straps of the headgear and the first and second lugs 786 and 794 provides a sufficient level of tension to resist movement of any of the structures of clip apparatus 724 and the headgear connected therewith for moving toward the eye of the patient, which is desirable.

The various embodiments thus advantageously enable a headgear and a mask to be removed from a patient and to be reinstalled thereon substantially without the need to individually remove straps of a headgear or to adjust them. Such configurations thus advantageously facilitate such removal and reinstallation of the improved respiratory patient interface device and improve comfort and fit to the patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A connection apparatus that is structured to extend between a mask and a headgear, the mask being structured to provide a flow of breathing gas to a patient, the headgear having a first strap and a second strap, at least a portion of the first strap being structured to overlie a region of the patient's head, at least a portion of the second strap being structured to overlie at least one other region of the patient's head, the connection apparatus comprising:
a clip apparatus comprising a having a first mount that is structured to be connected with the first strap and a second mount that is structured to be connected with the second strap; and
a face plate that is structured to overlie at least a portion of the mask, the face plate comprising an attachment apparatus having a connection upon which the clip is structured to be disposed, the connection comprising a first connection element and a second connection element, one of the first and second connection elements being a pivotable connection element that extends between the face plate and the clip and that is structured to enable the clip to be pivotable with respect to the face plate, at least a portion of the clip being removable from at least a portion of the connection.

2. (canceled)

3. The connection apparatus of claim 1, wherein the pivotable connection element is removable from at least one of the face plate and the clip and is structured to permit the face plate and the clip to be separated.

4. The connection apparatus of claim 1, wherein the other of the first and second connection elements is a fixed connection element that is structured to be movable between a first condition extending between the face plate and the clip and a second condition disconnected from at least one of the face plate and the clip, the fixed connection element in the first condition being structured to resist pivoting of the clip with respect to the face plate.

5. The connection apparatus of claim 1, wherein at least one of the face plate and the clip has a slot formed therein within which the pivotable connection element is slidably disposed.

6. The connection apparatus of claim 5, wherein the other of the first and second connection elements is a fixed connection element that is structured to be movable between a first condition extending between the face plate and the clip and a second condition disconnected from at least one of the face plate and the clip, the fixed connection element in the first condition being structured to resist pivoting of the clip with respect to the face plate; and wherein the slot comprises a seat that is structured to receive the pivotable connection element in the first condition of the fixed connection element.

7. The connection apparatus of claim 1, wherein the connection comprises a seat that is structured to removably receive at least a portion of the clip.

8. The connection apparatus of claim 7, wherein the first and second mounts are situated at alternate sides of the seat when the clip is received on the seat.

9. The connection apparatus of claim 8, wherein the clip comprises a finger grip situated anterior of the seat and the first and second mounts when the clip is received on the seat and the mask is situated on the patient.

10. The connection apparatus of claim 7, wherein the face plate comprises a resilient leg, the seat being situated at a free end of the leg, the leg being movable between a free state wherein the at least portion of the clip is removed from the seat and a deflected state wherein the at least portion of the clip is received in the seat, the seat in the free state of the leg being structured to be in a position situated anterior to the position of the seat in the deflected state with the mask situated on the patient.

11. The connection apparatus of claim 10, wherein the at least portion of the clip received in the seat is pivotably disposed in the seat.

12. The connection apparatus of claim 10, wherein the clip comprises a base that is structured to be received in the seat and a lug that is structured to extend in a direction posterior to the seat when the base that is received in the seat, the first and second mounts being situated on the lug at a location distal to the base.

13. The connection apparatus of claim 1, wherein the clip comprises a base that is structured to be disposed on the connection and a lug that is structured to extend in a direction posterior to the connection when the base is disposed on the connection and the mask is situated on the patient, at least one of the first mount and the second mount being situated on the lug at a location distal to the base.

14. The connection apparatus of claim 13, wherein the at least one of the first mount and the second mount is the one of the first mount and the second mount with which is connected the one of the first strap and the second strap that is disposed superior to the other of the first strap and the second strap when mask is situated on the patient.

15. The connection apparatus of claim 13, wherein the at least one of the first mount and the second mount is the one of the first mount and the second mount with which is connected the one of the first strap and the second strap that is disposed inferior to the other of the first strap and the second strap when mask is situated on the patient, and wherein the clip further comprises a flexible brace situated on the lug at a location distal to the base, the brace engaging the other of the first strap and the second strap and being structured to resist movement of the other of the first strap and the second strap in a direction toward the eye of the patient.

16. The connection apparatus of claim 15, wherein the other of the first strap and the second strap is longitudinally slidable with respect to the brace.

17-20. (canceled)
